(19) United States
(12) Patent Application Publication
(10) Pub. No.: US 2026/0275353 A1
(43) Pub. Date: Sep. 17, 2026

(54) COMPOUNDS TO TREAT INHERITED RETINAL DISEASE

(71) Applicant: UNIVERSITEIT GENT, Gent (BE)

(72) Inventors: Frauke Coppieters, Gent (BE); Alfredo Duenas Rey, Gent (BE); Manon Bouckaert, Gent (BE)

(21) Appl. No.: 19/473,454

(22) PCT Filed: Apr. 24, 2024
(86) PCT No.: PCT/EP2024/061297

§ 371 Date: Oct. 7, 2025

(30) Foreign Application Priority Data
Apr. 24, 2023 (EP) 23169591.7

(51) Int. Cl. C12N 15/113; A61K 9/00; A61K 31/7105; A61K 38/00; A61K 47/10; A61K 47/18; A61K 47/26; A61P 27/02; C12N 9/04; C12N 15/67

(52) U.S. Cl. CPC C12N 15/1137; A61K 9/0048; A61K 31/7105; A61K 47/10; A61K 47/183; A61K 47/26; A61P 27/02; C12N 9/0006; C12N 15/67; A61K 38/00; C12N 2310/11; C12N 2310/14; C12Y 101/013

(57) ABSTRACT

A field of treating inherited retinal disease (IRD). More specifically, the disclosure discloses a target region that is useful to screen for compounds capable of treating inherited retinal disease and discloses specific antisense oligonucleotides (ASOs) useful to treat the latter disease. This disclosure is based on the finding that the variant c.-123C>T in the 5′UTR of the RDH12 gene causes an upstream start codon (uATG) resulting in insufficient RDH12 protein production and hence causes inherited retinal disease. ASOs, which hybridize with a region surrounding or containing the uATG provide steric hindrance for recognition of the upstream start codon, thus redirecting the scanning ribosomes to the primary start codon, which results in increased RDH12 protein production.

### CROSS-REFERENCE TO RELATED APPLICATIONS

This application is a national phase entry under 35 U.S.C. § 371 of International Patent Application PCT/EP2024/061297, filed Apr. 24, 2024, designating the United States of America and published as International Patent Publication WO 2024/223696 A1 on Oct. 31, 2024, which claims the benefit under Article 8 of the Patent Cooperation Treaty of European Patent Convention patent application Ser. No. 23/169,591.7, filed Apr. 24, 2023.

### STATEMENT REGARDING ELECTRONIC FILING OF A SEQUENCE LISTING

Pursuant to 37 C.F.R. § 1.831 through 1.835, a Sequence Listing XML file entitled “P2022-113 PCT seqlisttxt,” 92,143 bytes in size, generated Sep. 30, 2025, has been submitted via EFS-Web is provided in lieu of a paper copy. This Sequence Listing is hereby incorporated by reference into the specification for its disclosures.

### TECHNICAL FIELD

This disclosure relates to the field of treating inherited retinal disease (IRD). More specifically, the disclosure discloses a target region that is useful to screen for compounds capable of treating IRD and discloses specific antisense oligonucleotides (ASOs) useful to treat the latter disease. This disclosure is based on the finding that the variant c.-123C>T in the 5′untranslated region (5′UTR) of the RDH12 gene causes an upstream start codon (uATG) resulting in insufficient RDH12 protein production and hence causes IRD. ASOs, which hybridize with a region spanning at least 50 nucleotides (nts) upstream and downstream of the uATG provide steric hindrance for recognition of the upstream start codon, thus redirecting the scanning ribosomes to the primary start codon, which results in increased RDH12 protein production.

### BACKGROUND

Inherited retinal diseases (IRDs) are a major cause of blindness affecting over 2 million people worldwide. IRDs are monogenic (each case is caused by mutations in a single gene), but extremely genetically heterogeneous. So far, many mutations in over 280 disease genes explain only 60% of cases, leaving a significant fraction molecularly unsolved.^{1,2} It is hypothesized that the remaining patients could be explained by mutations in non-coding regions of the IRD disease genes, which are not yet analyzed in a routine context. Indeed, recent studies have revealed an important contribution of non-coding variation in IRD including deep-intronic mis-splicing variants³⁻¹³ as well as variants affecting cis-regulatory regions.¹⁴⁻²⁰

The identification of genetic variants causing IRD is a prerequisite for genetic therapy.²¹ IRD is at the forefront of gene therapy, as illustrated by the recently approved gene augmentation therapy Luxturna. An alternative therapy focusses on antisense oligonucleotides (ASOs), short RNA/DNA molecules that mediate gene expression through either steric hindrance or RNA degradation. ASOs tackle several limitations associated with gene augmentation such as a limited cargo-capacity and challenging ocular delivery.²² In the IRD field, ASOs have so far been used to modulate splicing^{23,24} or induce RNA degradation²⁵. One example of a splice-modulating ASO strategy is described in WO2017/106370. The latter discloses a method to increase the expression of functional RNA by targeting retained-intron-containing pre-mRNA (RIC pre-mRNA), which is processed to non-productive RNA. WO2017/106370 describes the use of ASOs to target specific regions in the RIC pre-mRNA to splice out the retained intron and shift the synthesis of non-productive to productive mRNA, in this way increasing downstream protein production. This ASO strategy aims to increase protein production from a wild-type allele and is therefore especially beneficial for autosomal dominant diseases associated with haploinsufficiency, where patients carry one healthy copy and one mutant copy that produces either insufficient or nonfunctional protein.²⁶ Specifically, WO2017/106370 describes a region in intron 7 (=retained intron) of the RDH12 gene as an ASO target.

The ASO strategy described here is completely distinct from the method described in WO2017/106370, both in methodology and in application potential. First, WO2017/106370 describes ASOs that act at the pre-mRNA level to modulate splicing, whereas the approach described here includes ASOs that act at the mature RNA level to modulate protein translation by blocking ribosomal recognition of a novel upstream start codon (uATG). Furthermore, the ASOs described in WO2017/106370 are not expected to be beneficial for autosomal recessive RDH12-associated IRD (as targeted here), where two mutant alleles are present. As an example, applying the ASOs described in WO2017/106370 on the mutant allele containing the c.-123C>T variant, will still result in mRNA containing c.-123C>T and the associated upstream start codon, resulting in decreased protein translation.

The RDH12: c.-123C>T variant has previously been described in one database and two publications, none of which include the introduction of the novel uATG, nor the use of this region as a novel target for therapy:
1. In Clinvar, the variant has been submitted once as part of a predisposition screen in an ostensibly healthy population. Allele frequency data from public databases did not allow this variant to be ruled in or out of causing disease. Therefore, this variant was classified as a variant of unknown significance.
(ncbi.nlm.nih.gov/clinvar/variation/884100).
2. Thompson et al.27 described c.-123C>T as a rare sequence variant, considered not likely to be pathogenic. This study did not include functional analysis of this variant.
3. Cherry T et al.20 identified c.-123C>T, called “M2, chr14: 67,722,520 C>T (rs960563752)” in their study, in a patient with IRD (Leber Congenital Amaurosis or retinitis pigmentosa; no details provided). They hypothesize that this region represents an alternative promoter of RDH12, and hence a transcriptional effect of the variant. It is not mentioned nor suggested that c.-123C>T introduces an uATG and affects RDH12 translation. Cherry et al. cloned the alternative promoter (exon 1 of ENST00000267502.3 and an upstream sequence) upstream of a luciferase gene and showed that c.-123C>T decreases luciferase activity by 80%. Importantly, their experimental setup did not include comparison between mRNA levels and luciferase protein activity and was therefore not able to pinpoint at which level (transcription of translation) the effect occurs. In the disclosure, distinct luciferase assays where employed, which include the mRNA 5′UTR sequence (the non-coding part of exons 1, 2 and 3 of ENST00000551171) instead of the genomic promoter sequence, followed by downstream evaluation at both the mRNA and protein level, and show an effect only at the protein level. This was further confirmed with overexpression experiments. Taken together and in contrast to the study of Cherry et al., the disclosure indicates that c.-123C>T introduces an uATG into a strong Kozak consensus, predicted to create an uORF. Given that ribosomes would first encounter and start translating from this gained uATG, the disclosure shows that this variant decreases normal RDH12 translation and thus results in reduced protein levels, with unaltered RDH12 transcription.

### BRIEF DESCRIPTION OF THE DRAWINGS

FIG. 1: Results of a dual luciferase reporter assay, showing significantly reduced luciferase activity for the mutant (MT) construct compared to the wild-type (WT) construct.

FIGS. 2A and 2B: FIG. 2A) Overview of the mechanism of the c.-123C>T variant, which introduces a novel uATG in the 5′UTR of RDH12 that is predicted to result in translation of an out-of-frame uORF. Ribosomal recognition of the uATG results in reduced translation of the RDH12 pORF. FIG. 2B) The latter is confirmed by overexpression experiments in ARPE-19 cells of a construct containing the 5′UTR upstream of the RDH12 PORF. The mutant construct (c.-123C>T) does not affect mRNA levels (left panel), but results in significantly reduced RDH12 protein expression compared to the wild-type (WT) construct (middle and right panel).

FIG. 3: Evaluation of the wild-type 5′UTR (WT 5′UTR), the c.-123C>T variant (MT 5′UTR), and the p.Arg234His variant (MS), in cis with either the WT 5′UTR (WT 5′UTR+MS) or the MT 5′UTR (MT 5′UTR+MS), at the protein level through overexpression studies. The c.-123C>T variant results in decreased protein levels, whereas the p.Arg234His variant shows no effect at the protein level.

FIGS. 4A and 4B: Relative luminescence levels of cells transfected with either the mutant construct (FIG. 4A) or the wild-type construct (FIG. 4B) and ASOs, compared to cells transfected with only the mutant/wild-type construct. The ASOs are organized based on their target location (ASOs at the left are targeting a region upstream of the variant, while ASOs at the right are targeting a region downstream). Luminescence levels increase with up to 171% on the mutant construct (FIG. 4A). No or limited increases in luminescence level are observed for the wild-type construct (FIG. 4B).

FIGS. 5A and 5B: (FIG. 5A) Western blot result of free uptake of ASO36, ASO39 and ASO41 (6 μM) in patient-derived retinal organoids (D158). Relative protein levels compared to non-treated retinal organoids revealed an increase of 201% for ASO39. (FIG. 5B) As control, control retinal organoids were treated with the same ASOs, resulting in no increase at the protein level for ASO39.

### DETAILED DESCRIPTION

To further elucidate the role of non-coding variation in IRD and identify novel therapeutic targets, the 5′ untranslated region (5′UTR) of all currently known IRD disease genes was analyzed for the presence of mutations in a cohort of approximately 4.000 genetically unsolved IRD patients.

This disclosure relates to the identification of the c.-123C>T variant in the 5′UTR of the RDH12 gene (NM_152443.3) as a novel target region for genetic therapy. This variant was identified in patients with autosomal recessive IRD. More specifically, the disclosure discloses that this variant introduces an upstream start codon (uATG), predicted to result in an upstream open reading frame (uORF), and for which a reduced translation of the primary open reading frame (pORF) of RDH12 was shown through both luciferase and overexpression experiments. Furthermore, it was demonstrated that antisense oligonucleotides (ASOs) providing steric hindrance will block ribosomal recognition of this novel uATG, which results in increased RDH12 protein production.

This disclosure thus relates to a variant in the 5′UTR. 5′UTRs are major determinants of post-transcriptional control and translation efficiency that are located immediately upstream of the pORF and include the Kozak sequence around the initiation codon²⁸⁻³⁰. 5′UTRs harbor numerous cis-regulatory elements.³¹ A main example are uATGs and associated uORFs. Approximately half of mRNAs have at least one uATG and associated uORF in their 5′UTR.³² Ribosome recruitment at uORFs typically reduces translation of the pORF by up to 80%,³² There is a rising interest in uORFs in the context of human disease, as variants perturbing uORFs are an under-recognized mutation mechanism.³³

Disclosed here, the region of at least 50 nucleotides (nts) upstream and 50 nts downstream of the c.-123C>T variant represents a target for ASOs that block recognition of the novel uATG. ASOs blocking recognition of naturally occurring uORFs have been shown to increase translation of multiple human disease genes including CFTR, GADD³⁴ and RNASEH1, supporting the potential of this genetic therapy.³⁴⁻³⁶ The use of ASOs to treat a disease-causing variant introducing a novel uATG in the 5′UTR is unknown in the field of IRD and inherited disease in general.

This disclosure thus relates to the in vitro usage of a region including at least 50 nts upstream and 50 nts downstream of the c.-123C>T variant in the 5′UTR of the RDH12 gene as a target region to screen for molecules, which are capable to increase the translation of the RDH12 protein.

With the terms ‘a region including the variant c.-123C>T in the 5′UTR of the RDH12 gene’ is meant a region comprising (or consisting of) at least 50 nts upstream and 50 nts downstream of the upstream start codon in the 5′UTR of the RDH12 gene. This region is transcribed from DNA to RNA, but it is not translated to protein. The c.-123C>T nomenclature means that the C nucleotide that is located 123 nucleotides upstream from the start ATG codon changes into a T.

With the term ‘target region’ is meant the part of the 5′UTR region of RDH12 for which an ASO-tiling design includes the C nucleotide that is located 123 nucleotides upstream from the start ATG codon, and at least 50 nucleotides up and downstream sequence (of this C nucleotide).

With the term ‘to screen’ is meant to evaluate the potency of small molecules or ASOs overlapping the target region to increase translation of the pORF of RDH12. This evaluation is performed by ASO transfection or free uptake in cells/organoids, followed by evaluation of luminescence levels (luciferase assays) or protein levels through, for instance, Western blot or ELISA.

With the term ‘molecules’ or ‘compounds’ are meant ASOs or small molecules.

With the terms ‘capable to increase the translation of the RDH12 protein’ are meant a rescue of the reduction of protein levels exerted by the c.-123C>T variant by restoring the predominant recognition of the primary ATG start codon of RDH12 and hence normal RDH12 translation.

More specifically, the disclosure relates to the usage of a region including the variant c.-123C>T in the 5′UTR of the RDH12 gene as indicated above wherein the increased translation of the RDH12 protein results in the treatment of an inherited retinal disease.

Furthermore, the disclosure relates to the usage of a region including the variant c.-123C>T in the 5′UTR of the RDH12 gene as indicated above wherein the molecules are ASOs. These steric-hindrance ASOs bind to a complementary mRNA molecule providing steric hindrance for proteins (in this case ribosomal proteins). ASOs are different from primers and probes, which are also oligonucleotides but with different compositions/modifications and, importantly, a distinct purpose. ASOs are oligonucleotides designed to specifically hybridize with RNA molecules to modulate gene expression. Primers are short DNA oligonucleotides that are designed to bind (c) DN/RNA to amplify specific regions through the polymerase chain reaction (PCR), whereas probes are oligonucleotides used for the detection of specific DNA or RNA sequences.

This disclosure further discloses ASOs having the sequence provided in the table 1 below. It is clear for a skilled person that this list is not exhaustive, as more ASOs with different lengths and also covering at least the 50 nucleotides up and downstream of the uATG, can be used. It is further clear that also ASOs with a phosphorothioate backbone and different modifications such as (but not limited to): 2-O-Methyl, 2-O-Methoxyethyl, 2-O-ethyl, phosphorodiamidate morpholino can be used.

This disclosure further specifically discloses ASOs having the SEQ ID number ASO2, ASO35, ASO36, ASO38, ASO39, ASO40, ASO41 and ASO42 having the ability to significantly increase translation of the RDH12 protein.

TABLE 1
(SEQ ID NO: and associated RNA Sequence):

| | | | | | |
|---|---|---|---|---|---|
| 1 | CAUCCUGGGUGCUGAGCA | 35 | GCAGCAGCCUGACUCUG | 69 | GAGCAGAGCCCAGAAU |
| | | | A | | CC |
| 2 | CCAUCCUGGGUGCUGAGC | 36 | GAGCAGCAGCCUGACUC | 70 | UGAGCAGAGCCCAGAA |
| | | | U | | UC |
| 3 | UCCAUCCUGGGUGCUGAG | 37 | CUGAGCAGCAGCCUGAC | 71 | CUGAGCAGAGCCCAGA |
| | | | U | | AU |
| 4 | CUCCAUCCUGGGUGCUGA | 38 | UGCUGAGCAGCAGCCU | 72 | UCUGAGCAGAGCCCAG |
| | | | GA | | AA |
| 5 | UCUCCAUCCUGGGUGCUG | 39 | GGUGCUGAGCAGCAGC | 73 | CUCUGAGCAGAGCCCA |
| | | | CU | | GA |
| 6 | CUCUCCAUCCUGGGUGCU | 40 | UGGGUGCUGAGCAGCA | 74 | ACUCUGAGCAGAGCCC |
| | | | GC | | AG |
| 7 | CCUCUCCAUCCUGGGUGC | 41 | CCUGGGUGCUGAGCAG | 75 | GACUCUGAGCAGAGCC |
| | | | CA | | CA |
| 8 | UCCUCUCCAUCCUGGGUG | 42 | AUCCUGGGUGCUGAGC | 76 | UGACUCUGAGCAGAGC |
| | | | AG | | CC |
| 9 | CUCCUCUCCAUCCUGGGU | 43 | GCUUCUCUGCUCCUCUC | 77 | CUGACUCUGAGCAGAG |
| | | | C | | CC |
| 10 | GCUCCUCUCCAUCCUGGG | 44 | CUGCUUCUCUGCUCCUC | 78 | CCUGACUCUGAGCAGA |
| | | | U | | GC |
| 11 | UGCUCCUCUCCAUCCUGG | 45 | UGCUGCUUCUCUGCUCC | 79 | GCCUGACUCUGAGCAG |
| | | | U | | AG |
| 12 | CUGCUCCUCUCCAUCCUG | 46 | UCUGCUGCUUCUCUGCU | 80 | AGCCUGACUCUGAGCA |
| | | | C | | GA |
| 13 | UCUGCUCCUCUCCAUCCU | 47 | CUUCUGCUGCUUCUCUG | 81 | CAGCCUGACUCUGAGC |
| | | | C | | AG |
| 14 | CUCUGCUCCUCUCCAUCC | 48 | UGCUUCUGCUGCUUCUC | 82 | GCAGCCUGACUCUGAG |
| | | | U | | CA |
| 15 | UCUCUGCUCCUCUCCAUC | 49 | GCUGCUUCUGCUGCUUC | 83 | AGCAGCCUGACUCUGA |
| | | | U | | GC |
| 16 | UUCUCUGCUCCUCUCCAU | 50 | AGCAGCAGCCUGACUCU | 84 | CAGCAGCCUGACUCUG |
| | | | G | | AG |
| 17 | CAUCCUGGGUGCUGAGCA | 51 | UGAGCAGCAGCCUGAC | 85 | UGGCUGCUUCUGCUGC |
| | GC | | UC | | UU |
| 18 | CCAUCCUGGGUGCUGAGC | 52 | GCUGAGCAGCAGCCUG | 86 | UUGGCUGCUUCUGCUG |
| | AG | | AC | | CU |
| 19 | UCCAUCCUGGGUGCUGAG | 53 | GUGCUGAGCAGCAGCC | 87 | CUUGGCUGCUUCUGCU |
| | CA | | UG | | GC |
| 20 | CUCCAUCCUGGGUGCUGA | 54 | GGGUGCUGAGCAGCAG | 88 | UCUUGGCUGCUUCUGC |
| | GC | | CC | | UG |
| 21 | UCUCCAUCCUGGGUGCUG | 55 | CUGGGUGCUGAGCAGC | 89 | CUCUUGGCUGCUUCUG |
| | AG | | AG | | CU |
| 22 | CUCUCCAUCCUGGGUGCU | 56 | UCCUGGGUGCUGAGCA | 90 | GCUCUUGGCUGCUUCU |
| | GA | | GC | | GC |
| 23 | CCUCUCCAUCCUGGGUGC | 57 | CUUCUCUGCUCCUCUCC | 91 | AGCUCUUGGCUGCUUC |
| | UG | | A | | UG |
| 24 | UCCUCUCCAUCCUGGGUG | 58 | UGCUUCUCUGCUCCUCU | 92 | CAGCUCUUGGCUGCUU |
| | CU | | C | | CU |
| 25 | CUCCUCUCCAUCCUGGGU | 59 | GCUGCUUCUCUGCUCCU | 93 | CCAGCUCUUGGCUGCU |
| | GC | | C | | UC |
| 26 | GCUCCUCUCCAUCCUGGG | 60 | CUGCUGCUUCUCUGCUC | 94 | UCCAGCUCUUGGCUGC |
| | UG | | C | | UU |
| 27 | UGCUCCUCUCCAUCCUGG | 61 | UUCUGCUGCUUCUCUGC | 95 | CUCCAGCUCUUGGCUGC |
| | GU | | U | | U |
| 28 | CUGCUCCUCUCCAUCCUGG | 62 | GCUUCUGCUGCUUCUCU | 96 | GCUCCAGCUCUUGGCU |
| | G | | G | | GC |
| 29 | UCUGCUCCUCUCCAUCCUG | 63 | CUGCUUCUGCUGCUUCU | 97 | GGCUCCAGCUCUUGGC |
| | G | | C | | UG |
| 30 | CUCUGCUCCUCUCCAUCCU | 64 | GGCUGCUUCUGCUGCU | 98 | UGGCUCCAGCUCUUGG |
| | G | | UC | | CU |
| 31 | UCUCUGCUCCUCUCCAUCC | 65 | AGAGCCCAGAAUCCUA | 99 | CUGGCUCCAGCUCUUG |
| | U | | UU | | GC |
| 32 | UUCUCUGCUCCUCUCCAUC | 66 | CAGAGCCCAGAAUCCUA | 100 | UCUGGCUCCAGCUCUU |
| | C | | U | | GG |
| 33 | CUUCUCUGCUCCUCUCCAU | 67 | GCAGAGCCCAGAAUCCU | 101 | GUCUGGCUCCAGCUCU |
| | C | | A | | UG |
| 34 | GCUUCUCUGCUCCUCUCCA | 68 | AGCAGAGCCCAGAAUCC | 102 | GGUCUGGCUCCAGCUC |
| | U | | U | | UU |
| | | | | 103 | UGGUCUGGCUCCAGCU |
| | | | | | CU |
| | | | | 104 | CUGGUCUGGCUCCAGC |
| | | | | | UC |

This disclosure further discloses ASOs for use to treat an IRD and to ASOs hybridizing with the region surrounding an upstream start codon (uATG) caused by the variant c.-123C>T in the 5′UTR of the RDH12 gene for use to treat an IRD.

### Examples

### Methods

### Functional Evaluation of the Novel Therapeutic Target

### Cloning and Mutagenesis

For the luciferase constructs, a gBlocks™ gene fragment (IDT) consisting of the wild-type 5′UTR of RDH12 fused to a part of the Renilla luciferase reporter gene was cloned into a psiCHECK-2 dual luciferase vector (Promega) using the Cold Fusion cloning kit (Sanbio BV). Prior to cloning, the psiCHECK-2 vector was linearized by the restriction enzymes Nhel and AatII. For the generation of the overexpression construct, a gBlocks™ fragment was designed comprising the wild-type 5′UTR and coding sequence of RDH12. The sequence was modified to include a FLAG tag to evaluate translation and RDH12 protein levels. This fragment was then cloned into a pcDNA™3.1 (+) (Invitrogen) vector by restriction-ligation cloning and the recombinant vectors were then amplified in One Shot TOP10 Chemically Competent E. coli cells (Invitrogen) and purified using the NucleoBond Xtra Midi kit (Filter Service S.A).

The 5′UTR variant (c.-123C>T) and the missense variant (c.701G>A) were introduced using the Q5 Site-Directed Mutagenesis Kit (NEB) using variant-specific primers designed with the NEBaseChanger tool. The sequence of the inserts was confirmed by both Sanger sequencing using the BigDye Terminator v3.1 kit (Life Technologies) and long-read whole plasmid sequencing (Plasmidsaurus or Eurofins).

### Cell Culture

ARPE-19 (ATCC, CRL-2302) cells were grown in DMEM/F12 medium (Life Technologies) supplemented with 10% FBS (Pan-Biotech), 1% Penicillin/Streptomycin (Life Technologies), 1% non-essential amino acid solution (Life Technologies) and 0.1% amphotericin B (Life Technologies). Cells were cultured at 37° C. and 5% CO₂ and tested for mycoplasma contamination prior to use.

### Luciferase Reporter Assays

ARPE-19 cells were seeded in a 24-well plate (Greiner Bio-One) at a density of 50.000 cells/well in 1 ml of medium without antibiotics. The next day, cells were transfected at a 3:1 reagent: plasmid DNA ratio using TransIT-X2 Dynamic Delivery System (Mirus Bio) according to the manufacturer's instructions. After 24 hours (h), cells were lysed (Glo lysis buffer 1×) and luciferase activity was detected using the Dual-Glo Luciferase Assay System (Promega) in a Glomax 96-Microplate luminometer (Promega). Each transfection was performed in triplicate and each experiment was repeated at least three times to ensure reproducibility. For each well, the ratio of Renilla luciferase activity was normalized to Firefly luciferase activity. A custom R script was used to evaluate the effect of each variant on luciferase activity through a linear mixed effects model (implemented in the lme4 package37) including the luciferase vector as fixed effect and the biological replicate as random effect.

RNA isolation and quantitative polymerase chain reaction (qPCR)

Total RNA was extracted using the RNEASY MINI KIT® (Qiagen) according to the manufacturer's instructions. Isolated RNA underwent DNase treatment (ArcticZymes, Tromsø, Norway) prior to cDNA synthesis with the iScript cDNA Synthesis Kit (Bio-Rad Laboratories). For each cDNA sample, assays were prepared using SsoAdvanced Universal SYBR Green Supermix (Bio-Rad Laboratories) and run on LightCycler 480 System (Roche). Data were analyzed with qBase+38 and normalized to the Firefly luciferase expression. Statistical analyses were performed in R using the Wilcoxon rank sum test.

### Overexpression and Immunoblotting

To perform immunoblotting of RDH12, ARPE-19 cells were seeded in 12-well plates (Greiner Bio-One BVBA) at a density of 100,000 cells/well, allowed to settle overnight, and then transfected with the wild-type and mutant RDH12 overexpression vectors using the TRANSIT-X2® Dynamic Delivery System (Mirus Bio) according to the manufacturer's instructions. The pcDNA™3.1 (+) (Invitrogen) backbone vector was transfected as negative control. Four hours prior to RNA and protein isolation, cells were treated with 10 μM MG-132 proteasome inhibitor (Merck Life Science). For total protein extraction, cells were lysed with RIPA Buffer (Sigma-Aldrich) including protease inhibitory cocktail (Roche Diagnostics), phosphatase inhibitory cocktail 2, and phosphatase inhibitory cocktail 3 (Sigma-Aldrich). After centrifugation and reduction with 1M DTT (Sigma-Aldrich), protein lysates were subjected to sodium dodecyl sulfate-polyacrylamide gel electrophoresis using either NuPAGE™ 4-12% Bis-Tris Protein Gels (Fisher Scientific) with a ladder (Precision Plus Protein All Blue Standards, Bio-Rad Laboratories). Proteins were then transferred to a nitrocellulose membrane using the iBlot 2 Dry Blotting System (Thermo Fisher Scientific). Membranes were blocked for 2 hours at room temperature in 2% ECL™ Blocking Agent (Cytiva Amersham) and incubated at 4° C. overnight with an anti-FLAG (1:1000, F1804, Merck Life Science) primary antibody. Membranes were subsequently incubated for 2 hours at room temperature with the appropriate horseradish-peroxidase-conjugated secondary antibody (1:2500, 7076S, Cell Signaling Technologies) and revealed with the SuperSignal™ West Dura Extended Duration Substrate (Fisher Scientific). Membranes were scanned with an Amersham Imager 680 system (GE Healthcare Life Sciences). Protein quantification was performed by firstly stripping the membranes with Restore™ PLUS Western Blot Stripping Buffer (Thermo Scientific), incubating them for 1 hour at room temperature with a primary antibody against β-tubulin (1:2500, ab6046, Abcam) and for 2 hours with a horseradish-peroxidase-conjugated secondary antibody (1:2500, 7074S, Cell Signaling Technologies). RDH12 (FLAG) signal intensity quantification was achieved using ImageJ (NIH, v. 1.50i) and normalized to the amount of β-tubulin.

### Design and Evaluation of ASOs Targeting the Therapeutic Target Region

The effect of ASOs targeting the region containing and surrounding the c.-123C>T variant is evaluated using two strategies:

ASO (co)-transfection in luciferase assays, where the ASO effect is evaluated by quantification of the luciferase signal;

ASO addition to patient-derived retinal organoids, where the ASO effect is evaluated by quantification of RDH12 protein levels using immunoblotting; and

In addition to these strategies, the ASOs can also be evaluated in other cellular models. One example would be a relevant cell line (e.g., derived from retinal pigment epithelium) in which mutant RDH12 (containing the c.-123C>T variant) is incorporated through, for instance, lentiviral transduction of vectors containing mutant RDH12 cDNA.

### Antisense Oligonucleotide (ASO) Design

As an example, a region of 50 nts upstream and 50 nts downstream of the variant was evaluated and used for an ASO tiling design (see table 1 above), comprising ASOs with lengths of 18 and 20 nucleotides. In total, 28 ASOs were selected spanning the target region for in vitro evaluation. They all have a length of 18 nucleotides and contain a phosphorothioate backbone and 2-O-Methyl modifications. In addition, control ASOs could be designed representing either scrambled ASOs or mismatch ASOs. Scrambled ASOs are ASOs containing the same nucleotides as a target ASO but in a different order. Mismatch ASOs involve introducing multiple mismatches into the target ASO to assess the hybridization capability of the effective ASOs. Notably, other ASO lengths, positions (either within or outside the 50 nucleotides up and downstream of the variant), and modifications can be evaluated such as—but not restricted to -2-O-Methoxyethyl, 2-O-Ethyl, and phosphorodiamidate morpholino.

### Co-Transfection Experiments (Luciferase Assay)

In order to evaluate the ASOs, a co-transfection set-up was established. ARPE-19 cells were seeded in medium without antibiotics (50.000 cells/well). After 4 h, 75 ng of the mutant luciferase vector was transfected with TransIT-X2 at a 3/1 transfection reagent/DNA ratio. 24 h after seeding, medium was removed and cells were transfected with 150 nM of ASO with TransIT-X2 at a 3/1 transfection reagent/DNA ratio. Subsequently, a luciferase read-out was performed 24 h after ASO transfection. As negative control, all ASOs were also co-transfected with the wild-type vector. The most performant ASOs were selected for downstream evaluation in patient-derived retinal organoids. This setup also allows evaluation of ASO concentrations different from 150 nM. Statistical testing was performed using Graphpad Prism 10.2.1. The Mann-Whitney test was used as statistical test.

### Patient Material

Fresh blood samples were collected from one affected individual who is compound heterozygous for p. (Ala269Glyfs*2) and c. [-123C>T;701G>A]. c.701G>A (p.Arg234His) encodes for a hypomorphic missense variant always located in cis with c.-123C>T. Subsequently, induced pluripotent stem cells (iPSCs) were generated by the Erasmus MC iPSC Core Facility (Rotterdam, the Netherlands) using the CytoTune™-iPS 2.0 Sendai Reprogramming Kit (Invitrogen) from human erythroid progenitor cells (isolated from fresh blood). The iPSCs were evaluated by qPCR expression analysis and immunofluorescence of stem cell markers followed by trilineage differentiation and CNV-Seq (structural variant detection).

Retinal organoid differentiation and ASO treatment iPSCs from one patient and one control were differentiated into retinal organoids as described in Hallam et al.39 Briefly, iPSCs were seeded in a 96-well format in mTeSR plus (Stemcell Technologies) and 10 μM RockI (Selleckchem). After two days, a first differentiation medium was added to the cells containing 41% IMDM (Life Technologies), 41% HAM's F12 (Life Technologies), 15% Knock-out serum replacement (Life Technologies), 1% GlutaMAX (Life Technologies), 1% Chemically defined lipid concentrate (Life Technologies), 1% Penicillin/Streptomycin (Life Technologies) and 225 μM 1-Thioglycerol (Sigma-Aldrich). At day 6, 1.5 nM BMP4 (R&D) was added to the medium. From day 18, a second differentiation medium was added consisting of DMEM/F12 (Life Technologies), 10% FBS (Life Technologies), 1% GlutaMAX, 1% N2 (Life Technologies), 0.5 μM retinoic acid (Sigma-Aldrich), 0.1 mM taurine (Sigma-Aldrich), 1% penicillin/streptomycin and 0.25 μg/ml amphotericin B (Life Technologies). Two times a week, half of the medium was refreshed. From day 120, retinoic acid was removed from the medium. At day 140, half of the medium was removed and replaced by medium containing 6 μM of ASO. Half of the medium was changed two times a week. At day 157, retinal organoids were collected for protein analysis (after 17 days of treatment).

Notably, ASO transfection can also be performed using different ASOs, different ASO concentrations, different dosing regimes (e.g., repetitive dosing versus wash-out regimen), different retinal organoid differentiation protocols⁴⁰, and at different time points during the retinal organoid differentiation.

### Protein Extraction on Retinal Organoids

Ten retinal organoids were pooled per protein extraction. The organoids were washed with 1×PBS. After washing, 100 μl of RIPA lysis buffer was added containing 4% protease inhibitor (Sigma-Aldrich), 1% phosphatase inhibitor cocktail 2 (Sigma-Aldrich) and 1% phosphatase inhibitor cocktail 3 (Sigma-Aldrich). The organoids were homogenized by using a cell pellet mixer followed by 30 minutes incubation at 4° C. Subsequently, the samples were centrifuged for 10 min at 8000 RCF at 4° C. The supernatant was collected and protein concentrations were measured with the BCA protein assay kit (Life Technologies).

### Immunoblotting

14 μg of protein was loaded together with 1× reducing sample buffer (Life Technologies) and 2 μl of Dithiothreitol (DTT) in a precast 4-12% Bis-Tris gel (Life Technologies). Before loading, the samples were denatured for 5 min at 98° C. The gels were run at 120V in (3-(N-morpholino) propanesulfonic acid (MOPS) running buffer. The gels were then transferred to a nitrocellulose membrane (Life Technologies) by dry blotting using iBlot 2 (Life Technologies) for 7 min at 20V. Subsequently, the blots were blocked in 5% ECL blocking agent (Cytiva Amersham) for 2 h at room temperature and incubated at 4° C. overnight with the anti-RDH12 ( 1/500, Proteintech Europe) or anti-vinculin ( 1/2500, Bioké) primary antibodies. The next day, the blots were washed with 1× TBST followed by incubation of a horseradish-peroxidase-conjugated anti-rabbit secondary antibody ( 1/2500, Cell Signaling) for 2 h at room temperature. After washing, the blots were developed using the West Dura Extended Duration Substrate (Life Technologies). The membranes were scanned with an Amersham Imager 680 system (GE Healthcare Life Sciences). Protein quantification was performed using ImageJ and normalized to vinculin.

### Results

### 1. Functional Evaluation of the Novel Therapeutic Target

A large-scale analysis of all variants located in the 5′UTR of 1,682 in-house IRD exomes and 2,397 IRD genomes of the 100,000 Genomes Project (Genomics England, GE) was performed. This resulted in the identification of 51 variants predicted to create a novel uORF, one of which is c.-123C>T in the RDH12 gene (NM_152443.3). This variant was found in one case from the GE cohort with bi-allelic RDH12 coding variants (c. [701G>A]; [c.735_743del]). This variant was subsequently identified in 8 additional RDH12 bi-allelic patients from 6 autosomal recessive IRD families worldwide through evaluation of the in-house IRD cohort and collaborations with C. Rivolta (Basel, Switzerland) and C. Ayuso (Madrid, Spain).

Interestingly, the c.-123C>T variant was always found located in cis with p.Arg234His. This finding has not yet been described before. The pathogenicity of p.Arg234His has been questioned in view of functional assays performed by Thompson et al., revealing RDH12 protein levels and catalytic activity comparable to the wild-type or polymorphic alleles, respectively.²⁷ As none of the existing publications describe either the mechanism of introducing a novel uATG nor its cis-configuration with p.Arg234His, an overlooked pathogenic role of the RDH12: c.-123C>T variant in IRD is disclosed.

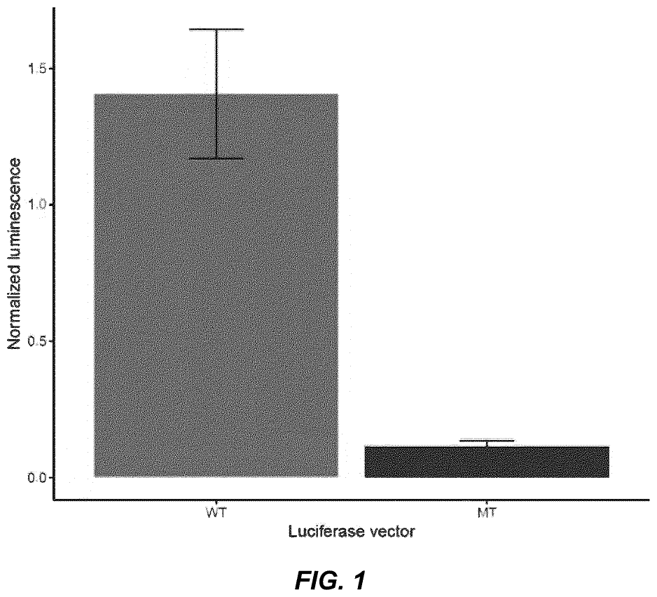

It is shown that the c.-123C>T variant introduces a novel uATG with a strong Kozak sequence, which is predicted to result in translation of a novel uORF of 25 amino acids. This uORF is located upstream and out-of-frame with respect to the RDH12 PORF. It is disclosed that recognition of this novel uATG results in decreased translation of the RDH12 pORF, a mechanism that could be amenable to ASO-mediated therapeutic intervention. To support this finding, dual luciferase reporter assays were performed in which the 5′UTR of RDH12 was cloned upstream of the Renilla luciferase gene. Introduction of the c.-123C>T variant resulted in a significant decrease in luciferase expression (91% decrease, p<0.001) compared to the wild-type construct (FIG. 1). Importantly, qPCR-based mRNA expression analysis revealed that relative Renilla luciferase mRNA levels remain unchanged, showing an effect solely at the translational level.

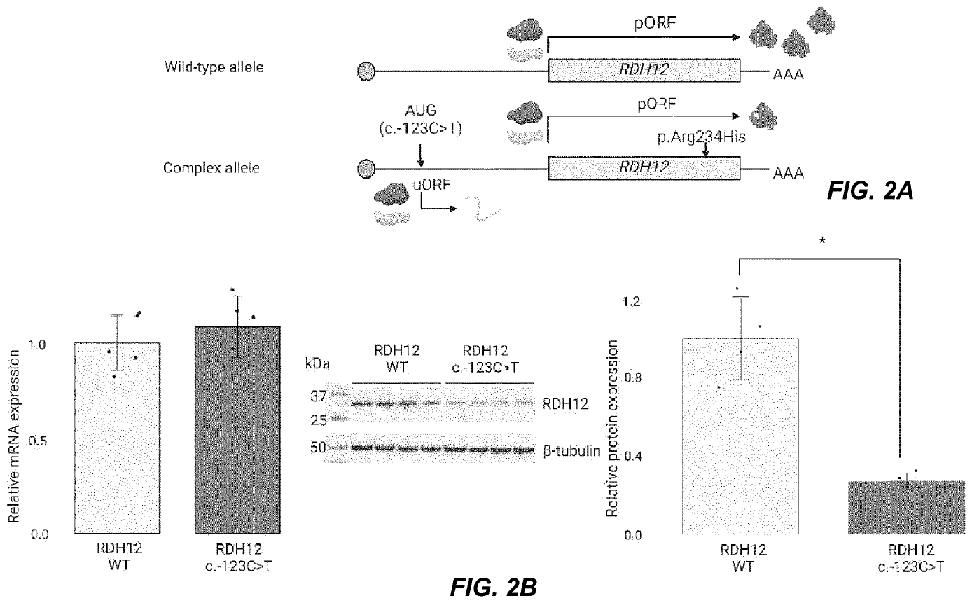

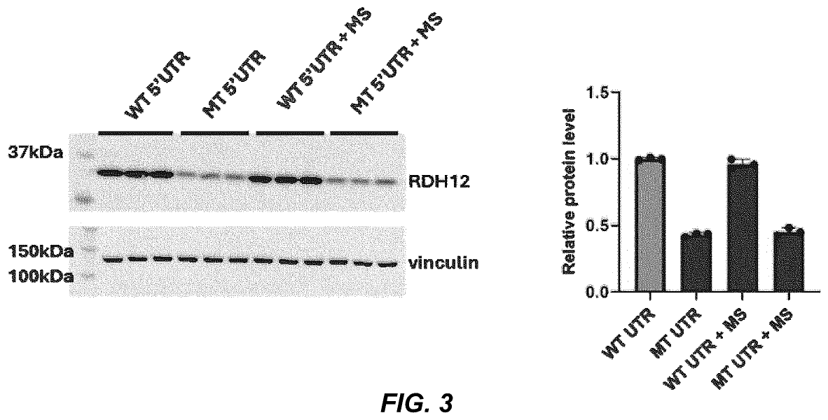

In view of these results and the predicted 5′UTR variant consequence of introducing a uATG and uORF, the potentially exclusive effect at the translational level was further inspected by assessing RDH12 mRNA abundance and protein levels in an overexpression setting in ARPE-19 cells. To this end, an overexpression vector containing the 5′UTR cloned upstream of the RDH12 pORF was used. By immunoblotting and RT-qPCR, unaltered mRNA, but significantly (p<0.05) reduced RDH12 protein levels for the mutant 5′UTR construct compared to the wild-type 5′UTR construct (FIGS. 2A, 2B) was observed. This provides further evidence for a post-transcriptional or translational effect, already shown by the luciferase assays. Furthermore, overexpression experiments were also performed to determine the effect of the missense variant (p.Arg234His) located in cis with the 5′UTR variant. No effect at protein level was observed when the missense variant was present, indicating that solely the 5′UTR variant affects protein translation (FIG. 3). Overall, the results indicate that not p.Arg234His alone, but rather the combination of p.Arg234His and c.-123C>T, or c.-123C>T by itself, is disease-causing.

### 2. Design and Evaluation of ASOs Targeting the Therapeutic Target Region

Finally, it is disclosed that recognition of the novel uATG can be blocked by ASO treatment, resulting in restoration of RDH12 protein expression to levels compatible with normal function. To this end, additional experiments have been performed to evaluate ASOs using two strategies. First, ASOs were evaluated in the existing luciferase assays in order to evaluate if ASOs increase luciferase expression and activity. Next, a selection of the best-performing ASOs were evaluated in a relevant patient-derived retinal model. As RDH12 expression is limited to (inaccessible) photoreceptor cells, induced pluripotent stem cells (iPSCs) were generated from fresh blood samples. Next, iPSCs were differentiated to retinal organoids, to which ASOs were added (through gymnosis), followed by evaluation of RDH12 protein levels.

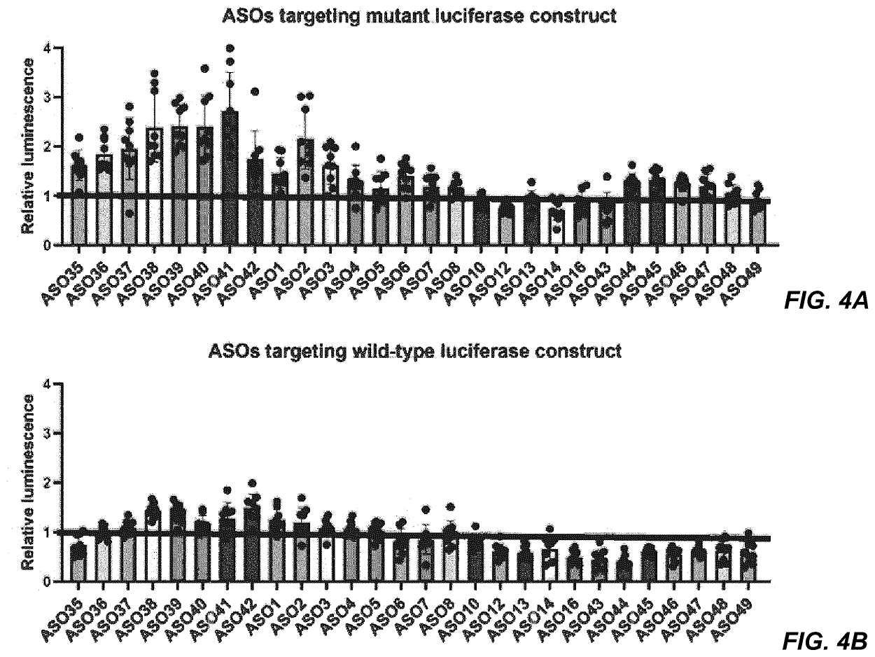

In total, twenty-eight ASOs were evaluated in a co-transfection luciferase set-up in ARPE-19 cells. The highest increase observed was 171% (ASO41). Different ASOs (ASO2, 35, 36, 38, 39, 40, 41, 42) showed significant increases in luminescence level (p<0,0001). (FIG. 4A). Remarkably, ASOs targeting a region upstream of the 5′UTR variant resulted in higher luminescence levels, highlighting a location-dependent effect of the ASOs. As a control, all ASOs were also evaluated on the wild-type luciferase construct (FIG. 4B). In theory, no effect should be seen as there is no uATG and as such no ribosomal binding. Upon transfection, limited to no increases were observed on the wild-type construct, supporting a specific effect of the ASOs on the mutant construct.

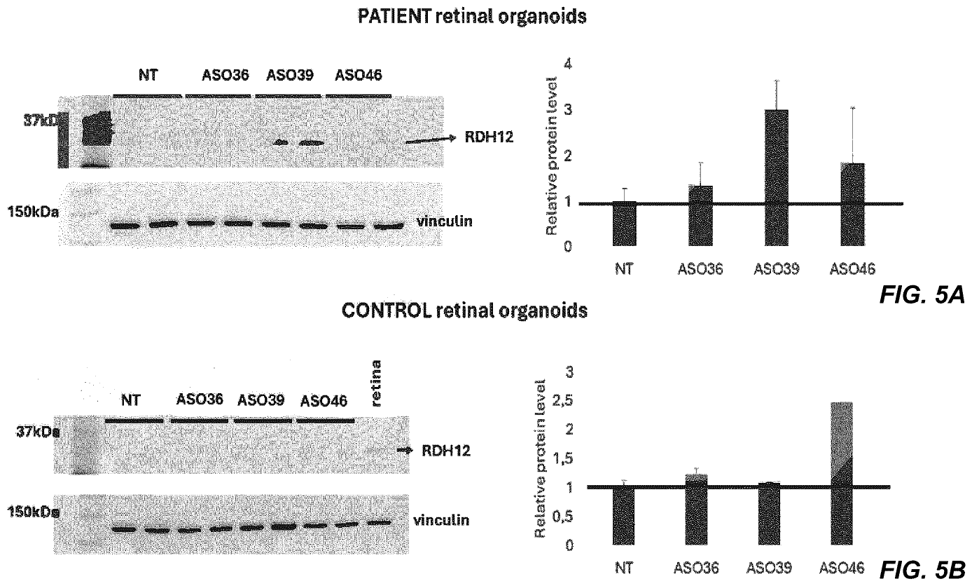

Next, ASO36, 39 and 46 were selected for evaluation in patient-derived retinal organoids. ASO36 (85% (p<0.0001)) and ASO39 (142% (p<0.0001)) resulted in significantly increase luminescence levels with limited technical variation, and are located at different positions upstream of the variant. ASO46 resulted in no significant increase (p=0.0244) and is located in a region downstream of the 5′UTR variant. Upon gymnosis of these ASOs in patient-derived retinal organoids, ASO39 resulted in a protein increase of 201% (FIG. 5A). As a control, control retinal organoids were also treated with the above-mentioned ASOs, resulting in no increase in protein level (except for ASO46, however only one replicate was available). In general, ASO46 showed high variability in the experiments on retinal organoids. (FIG. 5B).

To further evaluate whether these ASOs have a functional effect and the ability to ameliorate the IRD phenotype, functional tests can be performed in patient-derived retinal organoids. One example would be the evaluation of the conversion of all-trans retinal through enzymatic assays through, for instance, normal-phase high performance liquid chromatography41 or fluorescence life imaging microscopy42.

### REFERENCES

1 The Retinal Information Network. 1996-2023 sph.uth.edu/retnet.
2. Farrar, G. J. et al. Toward an elucidation of the molecular genetics of inherited retinal degenerations. Hum Mol Genet 26, R2-R11 (2017).
3. Fadaie, Z. et al. Whole genome sequencing and in vitrosplice assays reveal genetic causes for inherited retinal diseases. NPJ Genom Med 6, 97 (2021).
4. Liquori, A. et al. Whole USH2A Gene Sequencing Identifies Several New Deep Intronic Mutations. Hum Mutat 37, 184-193 (2016).
5. Holtan, J. P., Selmer, K. K., Heimdal, K. R. & Bragadóttir, R. Inherited retinal disease in Norway-a characterization of current clinical and genetic knowledge. Acta Ophthalmol 98, 286-295 (2020).
6. Reurink, J. et al. Whole genome sequencing for USH2A-associated disease reveals several pathogenic deep-intronic variants that are amenable to splice correction. Human Genetics and Genomics Advances 4, 26 (2023).
7. Bauwens, M. et al. ABCA4-associated disease as a model for missing heritability in autosomal recessive disorders: novel noncoding splice, cis-regulatory, structural, and recurrent hypomorphic variants. Genetics in Medicine 21, 1761 (2019).
8. Sangermano, R. et al. Deep-intronic ABCA4 variants explain missing heritability in Stargardt disease and allow correction of splice defects by antisense oligonucleotides. Genet Med 21, 1751-1760 (2019).
9. Khan, M. et al. Resolving the dark matter of ABCA4 for 1054 Stargardt disease probands through integrated genomics and transcriptomics. Genetics in Medicine 22, 1235-1246 (2020).
10. Daich Varela, M. et al. Multidisciplinary team directed analysis of whole genome sequencing reveals pathogenic non-coding variants in molecularly undiagnosed inherited retinal dystrophies. Hum Mol Genet 32, 595-607 (2023).
11. Qian, X. et al. Identification of Deep-Intronic Splice Mutations in a Large Cohort of Patients With Inherited Retinal Diseases. Front Genet 12, 647400 (2021).
12. Weisschuh, N. et al. Deep-intronic variants in CNGB3 cause achromatopsia by pseudoexon activation. Hum Mutat 41, 255 (2020).
13. Jamshidi, F. et al. Contribution of non-coding mutations to RPGRIP1-mediated inherited retinal degeneration. Genet Med 21, 694 (2019).
14. Bauwens, M. et al. ABCA4-associated disease as a model for missing heritability in autosomal recessive disorders: novel noncoding splice, cis-regulatory, structural, and recurrent hypomorphic variants. Genet Med 21, 1761-1771 (2019).
15. Coppieters, F. et al. Hidden Genetic Variation in LCA9-Associated Congenital Blindness Explained by 5′UTR Mutations and Copy-Number Variations of NMNAT1. Hum Mutat 36, 1188 (2015).
16. Daich Varela, M. et al. Multidisciplinary team directed analysis of whole genome sequencing reveals pathogenic non-coding variants in molecularly undiagnosed inherited retinal dystrophies. Hum Mol Genet 32, 595-607 (2023).
17. Ruberto, F. P. et al. Heterozygous deletions of noncoding parts of the PRPF31 gene cause retinitis pigmentosa via reduced gene expression. Mol Vis 27, 107 (2021).
18. Van de Sompele, S. et al. Multi-omics approach dissects cis-regulatory mechanisms underlying North Carolina macular dystrophy, a retinal enhanceropathy. Am J Hum Genet 109, 2029-2048 (2022).
19. Small, K. W. et al. North Carolina Macular Dystrophy is caused by dysregulation of the retinal transcription factor PRDM13. Ophthalmology 123, 9 (2016).
20. Cherry, T. J. et al. Mapping the cis-regulatory architecture of the human retina reveals noncoding genetic variation in disease. Proc Natl Acad Sci USA 117, 9001-9012 (2020).
21. Auricchio, A., Smith, A. J. & Ali, R. R. The Future Looks Brighter After 25 Years of Retinal Gene Therapy. home.liebertpub.com/hum 28, 982-987 (2017).
22. Girach, A. et al. RNA-based therapies in inherited retinal diseases. Ther Adv Ophthalmol 14, 251584142211346 (2022).
23. Dulla, K. et al. Splice-Modulating Oligonucleotide QR-110 Restores CEP290 mRNA and Function in Human c.2991+1655A>G LCA10 Models. Mol Ther Nucleic Acids 12, 730 (2018).
24. Dulla, K. et al. Antisense oligonucleotide-based treatment of retinitis pigmentosa caused by USH2A exon 13 mutations. Molecular Therapy 29, 2441-2455 (2021).
25. Murray, S. F. et al. Allele-Specific Inhibition of Rhodopsin With an Antisense Oligonucleotide Slows Photoreceptor Cell Degeneration. Invest Ophthalmol Vis Sci 56, 6362 (2015).
26. Lim, K. H. et al. Antisense oligonucleotide modulation of non-productive alternative splicing upregulates gene expression. Nat Commun 11, (2020).
27. Thompson, D. A. et al. Retinal degeneration associated with RDH12 mutations results from decreased 11-cis retinal synthesis due to disruption of the visual cycle. Hum Mol Genet 14, 3865-3875 (2005).
28. Jackson, R. J., Hellen, C. U. T. & Pestova, T. V. THE MECHANISM OF EUKARYOTIC TRANSLATION INITIATION AND PRINCIPLES OF ITS REGULATION. doi: 10.1038/nrm2838.
29. Araujo, P. R. et al. Before It Gets Started: Regulating Translation at the 5′ UTR. Comp Funct Genomics 2012, (2012).
30. Kozak, M. Influences of mRNA secondary structure on initiation by eukaryotic ribosomes. Proc Natl Acad Sci USA 83, 2850-2854 (1986).
31. Leppek, K., Das, R. & Barna, M. Functional 5′ UTR mRNA structures in eukaryotic translation regulation and how to find them. Nat Rev Mol Cell Biol 19, 158 (2018).
32. Calvo, S. E., Pagliarini, D. J. & Mootha, V. K. Upstream open reading frames cause widespread reduction of protein expression and are polymorphic among humans. Proceedings of the National Academy of Sciences 106, 7507-7512 (2009).
33. Whiffin, N. et al. Characterising the loss-of-function impact of 5′ untranslated region variants in 15,708 individuals. Nat Commun 11, 2523 (2020).
34. Liang, X. H. et al. Translation efficiency of mRNAs is increased by antisense oligonucleotides targeting upstream open reading frames. Nat Biotechnol 34, 875-880 (2016).
35. Sasaki, S. et al. Steric Inhibition of 5′ UTR Regulatory Elements Results in Upregulation of Human CFTR. Molecular Therapy 27, 1749-1757 (2019).
36. Kidwell, A. et al. Translation Rescue by Targeting Ppp1r15a through Its Upstream Open Reading Frame in Sepsis-Induced Acute Kidney Injury in a Murine Model. Journal of the American Society of Nephrology 34, 220-240 (2023).
37. Bates, D., Mächler, M., Bolker, B. M. & Walker, S. C. Fitting linear mixed-effects models using lme4. J Stat Softw 67, (2015).
38. Hellemans, J., Mortier, G., De Paepe, A., Speleman, F. & Vandesompele, J. Open Access Method qBase relative quantification framework and software for management and automated analysis of real-time quantitative PCR data. (2007) doi: 10.1186/gb-2007-8-2-r19.
39. Hallam, D. et al. Human-Induced Pluripotent Stem Cells Generate Light Responsive Retinal Organoids with Variable and Nutrient-Dependent Efficiency. Stem Cells 36, 1535 (2018).
40. Kurzawa-Akanbi, M. et al. Pluripotent stem cell-derived models of retinal disease: Elucidating pathogenesis, evaluating novel treatments, and estimating toxicity. Prog Retin Eye Res 101248 (2024) doi: 10.1016/j.preteyeres.2024.101248.
41. Sarkar, H., Toms, M. & Moosajee, M. Involvement of Oxidative and Endoplasmic Reticulum Stress in RDH12-Related Retinopathies. Int J Mol Sci 22, (2021).
42. Samimi, K. et al. In situ autofluorescence lifetime assay of a photoreceptor stimulus response in mouse retina and human retinal organoids. Biomed Opt Express 13, (2022).

1-7. (canceled)

8. An antisense oligonucleotide (ASO) comprising a polynucleotide that hybridizes under stringent conditions to a region of the RDH12 gene 5′ untranslated region (5′UTR) containing the variant c.-123C>T, wherein the ASO is capable of increasing translation of RDH12 protein.

9. The ASO of claim 8, wherein the ASO inhibits ribosomal recognition of an upstream start codon (uATG) caused by the variant c.-123C>T.

10. The ASO of claim 8, wherein the ASO consists of 12 to 25 nucleotides.

11. The ASO of claim 8, wherein the ASO comprises a polynucleotide selected from SEQ ID NOs: 1-104.

12. The ASO of claim 11, wherein the ASO comprises a polynucleotide selected from the group consisting of SEQ ID NOs: 2, 35, 36, 38, 39, 40, 41, and 42.

13. The ASO of claim 12, wherein the ASO increases translation of RDH12 protein in a cell comprising the variant c.-123C>T.

14. An antisense oligonucleotide (ASO) comprising means for increasing translation of RDH12 protein in a cell comprising variant c.-123C>T in the 5′UTR of the RDH12 gene,
wherein the means comprises a polynucleotide that hybridizes to the 5′UTR region containing the variant.

15. The ASO of claim 14, wherein the ASO comprises a polynucleotide selected from SEQ ID NOs: 1-104.

16. The ASO of claim 15, wherein the ASO comprises a polynucleotide selected from the group consisting of SEQ ID NOs: 2, 35, 36, 38, 39, 40, 41, and 42.

17. The ASO of claim 16, wherein the ASO increases translation of RDH12 protein in a cell comprising the variant c.-123C>T.

18. A pharmaceutical composition formulated for intravitreal administration, the pharmaceutical formulation comprising:
the ASO of claim 8 and
a pharmaceutically acceptable carrier.

19. A pharmaceutical composition formulated for intravitreal administration, the pharmaceutical formulation comprising:
the ASO of claim 14 and
a pharmaceutically acceptable carrier.

20. The pharmaceutical composition of claim 18, further comprising a stabilizing excipient selected from sugars, polyols, amino acids, and mixtures thereof.

21. The pharmaceutical composition of claim 19, further comprising a stabilizing excipient selected from sugars, polyols, amino acids, and mixtures thereof.

22. A method of treating an inherited retinal disease in a subject in need thereof, the method comprising:
administering to the subject the ASO of claim 8.

23. A method of treating an inherited retinal disease in a subject in need thereof, the method comprising:
administering to the subject the ASO of claim 14.

24. The method according to claim 22, wherein the ASO comprises a polynucleotide selected from the group consisting of SEQ ID NOs: 2, 35, 36, 38, 39, 40, 41, and 42.

25. The method according to claim 23, wherein the ASO comprises a polynucleotide selected from the group consisting of SEQ ID NOs: 2, 35, 36, 38, 39, 40, 41, and 42.

26. The method according to claim 23, wherein the ASO inhibits ribosomal recognition of an upstream start codon (uATG) caused by the variant c.-123C>T.

27. The method according to claim 23, wherein the ASO increases translation of RDH12 protein in the subject's retinal cells.

28. A method of increasing translation of RDH12 protein in vitro, the method comprising:
contacting a polynucleotide comprising the variant c.-123C>T in the 5′UTR of the RDH12 gene with the ASO of claim 8.

29. The method according to claim 28, wherein the ASO comprises a polynucleotide selected from the group consisting of SEQ ID NOs: 2, 35, 36, 38, 39, 40, 41, and 42.

30. The method according to claim 28, wherein the ASO increases translation of the RDH12 protein by at least 50% compared to a control.

31. A method of increasing translation of RDH12 protein in vitro, the method comprising:
contacting a polynucleotide comprising the variant c.-123C>T in the 5′UTR of the RDH12 gene with the ASO of claim 14.

32. A method of screening a molecule for the ability to increase translation of RDH12 protein, the method comprising:
(a) contacting the molecule with a polynucleotide comprising the variant c.-123C>T in the 5′ untranslated region (5′UTR) of the RDH12 gene; and
(b) detecting an increase in translation of the RDH12 protein.


SEQUENCE LISTING (104 sequences)

SEQ ID NO: 1 - RNA, 18 nt, synthetic construct
  source 1..18: mol_type = other RNA
catcctgggtgctgagca

SEQ ID NO: 2 - RNA, 18 nt, synthetic construct
  source 1..18: mol_type = other RNA
ccatcctgggtgctgagc

SEQ ID NO: 3 - RNA, 18 nt, synthetic construct
  source 1..18: mol_type = other RNA
tccatcctgggtgctgag

SEQ ID NO: 4 - RNA, 18 nt, synthetic construct
  source 1..18: mol_type = other RNA
ctccatcctgggtgctga

SEQ ID NO: 5 - RNA, 18 nt, synthetic construct
  source 1..18: mol_type = other RNA
tctccatcctgggtgctg

SEQ ID NO: 6 - RNA, 18 nt, synthetic construct
  source 1..18: mol_type = other RNA
ctctccatcctgggtgct

SEQ ID NO: 7 - RNA, 18 nt, synthetic construct
  source 1..18: mol_type = other RNA
cctctccatcctgggtgc

SEQ ID NO: 8 - RNA, 18 nt, synthetic construct
  source 1..18: mol_type = other RNA
tcctctccatcctgggtg

SEQ ID NO: 9 - RNA, 18 nt, synthetic construct
  source 1..18: mol_type = other RNA
ctcctctccatcctgggt

SEQ ID NO: 10 - RNA, 18 nt, synthetic construct
  source 1..18: mol_type = other RNA
gctcctctccatcctggg

SEQ ID NO: 11 - RNA, 18 nt, synthetic construct
  source 1..18: mol_type = other RNA
tgctcctctccatcctgg

SEQ ID NO: 12 - RNA, 18 nt, synthetic construct
  source 1..18: mol_type = other RNA
ctgctcctctccatcctg

SEQ ID NO: 13 - RNA, 18 nt, synthetic construct
  source 1..18: mol_type = other RNA
tcygctcctctccatcct

SEQ ID NO: 14 - RNA, 18 nt, synthetic construct
  source 1..18: mol_type = other RNA
ctctgctcctctccatcc

SEQ ID NO: 15 - RNA, 18 nt, synthetic construct
  source 1..18: mol_type = other RNA
tctctgctcctctccatc

SEQ ID NO: 16 - RNA, 18 nt, synthetic construct
  source 1..18: mol_type = other RNA
ttctctgctcctctccat

SEQ ID NO: 17 - RNA, 20 nt, synthetic construct
  source 1..20: mol_type = other RNA
catcctgggtgctgagcagc

SEQ ID NO: 18 - RNA, 20 nt, synthetic construct
  source 1..20: mol_type = other RNA
ccatcctgggtgctgagcag

SEQ ID NO: 19 - RNA, 20 nt, synthetic construct
  source 1..20: mol_type = other RNA
tccatcctgggtgctgagca

SEQ ID NO: 20 - RNA, 20 nt, synthetic construct
  source 1..20: mol_type = other RNA
ctccatcctgggtgctgagc

SEQ ID NO: 21 - RNA, 20 nt, synthetic construct
  source 1..20: mol_type = other RNA
tctccatcctgggtgctgag

SEQ ID NO: 22 - RNA, 20 nt, synthetic construct
  source 1..20: mol_type = other RNA
ctctccatcctgggtgctga

SEQ ID NO: 23 - RNA, 20 nt, synthetic construct
  source 1..20: mol_type = other RNA
cctctccatcctgggtgctg

SEQ ID NO: 24 - RNA, 20 nt, synthetic construct
  source 1..20: mol_type = other RNA
tcctctccatcctgggtgct

SEQ ID NO: 25 - RNA, 20 nt, synthetic construct
  source 1..20: mol_type = other RNA
ctcctctccatcctgggtgc

SEQ ID NO: 26 - RNA, 20 nt, synthetic construct
  source 1..20: mol_type = other RNA
gctcctctccatcctgggtg

SEQ ID NO: 27 - RNA, 20 nt, synthetic construct
  source 1..20: mol_type = other RNA
tgctcctctccatcctgggt

SEQ ID NO: 28 - RNA, 20 nt, synthetic construct
  source 1..20: mol_type = other RNA
ctgctcctctccatcctggg

SEQ ID NO: 29 - RNA, 20 nt, synthetic construct
  source 1..20: mol_type = other RNA
tctgctcctctccatcctgg

SEQ ID NO: 30 - RNA, 20 nt, synthetic construct
  source 1..20: mol_type = other RNA
ctctgctcctctccatcctg

SEQ ID NO: 31 - RNA, 20 nt, synthetic construct
  source 1..20: mol_type = other RNA
tctctgctcctctccatcct

SEQ ID NO: 32 - RNA, 20 nt, synthetic construct
  source 1..20: mol_type = other RNA
ttctctgctcctctccatcc

SEQ ID NO: 33 - RNA, 20 nt, synthetic construct
  source 1..20: mol_type = other RNA
cttctctgctcctctccatc

SEQ ID NO: 34 - RNA, 20 nt, synthetic construct
  source 1..20: mol_type = other RNA
gcttctctgctcctctccat

SEQ ID NO: 35 - RNA, 18 nt, synthetic construct
  source 1..18: mol_type = other RNA
gcagcagcctgactctga

SEQ ID NO: 36 - RNA, 18 nt, synthetic construct
  source 1..18: mol_type = other RNA
gagcagcagcctgactct

SEQ ID NO: 37 - RNA, 18 nt, synthetic construct
  source 1..18: mol_type = other RNA
ctgagcagcagcctgact

SEQ ID NO: 38 - RNA, 18 nt, synthetic construct
  source 1..18: mol_type = other RNA
tgctgagcagcagcctga

SEQ ID NO: 39 - RNA, 18 nt, synthetic construct
  source 1..18: mol_type = other RNA
ggtgctgagcagcagcct

SEQ ID NO: 40 - RNA, 18 nt, synthetic construct
  source 1..18: mol_type = other RNA
tgggtgctgagcagcagc

SEQ ID NO: 41 - RNA, 18 nt, synthetic construct
  source 1..18: mol_type = other RNA
cctgggtgctgagcagca

SEQ ID NO: 42 - RNA, 18 nt, synthetic construct
  source 1..18: mol_type = other RNA
atcctgggtgctgagcag

SEQ ID NO: 43 - RNA, 18 nt, synthetic construct
  source 1..18: mol_type = other RNA
gcttctctgctcctctcc

SEQ ID NO: 44 - RNA, 18 nt, synthetic construct
  source 1..18: mol_type = other RNA
ctgcttctctgctcctct

SEQ ID NO: 45 - RNA, 18 nt, synthetic construct
  source 1..18: mol_type = other RNA
tgctgcttctctgctcct

SEQ ID NO: 46 - RNA, 18 nt, synthetic construct
  source 1..18: mol_type = other RNA
tctgctgcttctctgctc

SEQ ID NO: 47 - RNA, 18 nt, synthetic construct
  source 1..18: mol_type = other RNA
cttctgctgcttctctgc

SEQ ID NO: 48 - RNA, 18 nt, synthetic construct
  source 1..18: mol_type = other RNA
tgcttctgctgcttctct

SEQ ID NO: 49 - RNA, 18 nt, synthetic construct
  source 1..18: mol_type = other RNA
gctgcttctgctgcttct

SEQ ID NO: 50 - RNA, 18 nt, synthetic construct
  source 1..18: mol_type = other RNA
agcagcagcctgactctg

SEQ ID NO: 51 - RNA, 18 nt, synthetic construct
  source 1..18: mol_type = other RNA
tgagcagcagcctgactc

SEQ ID NO: 52 - RNA, 18 nt, synthetic construct
  source 1..18: mol_type = other RNA
gctgagcagcagcctgac

SEQ ID NO: 53 - RNA, 18 nt, synthetic construct
  source 1..18: mol_type = other RNA
gtgctgagcagcagcctg

SEQ ID NO: 54 - RNA, 18 nt, synthetic construct
  source 1..18: mol_type = other RNA
gggtgctgagcagcagcc

SEQ ID NO: 55 - RNA, 18 nt, synthetic construct
  source 1..18: mol_type = other RNA
ctgggtgctgagcagcag

SEQ ID NO: 56 - RNA, 18 nt, synthetic construct
  source 1..18: mol_type = other RNA
tcctgggtgctgagcagc

SEQ ID NO: 57 - RNA, 18 nt, synthetic construct
  source 1..18: mol_type = other RNA
cttctctgctcctctcca

SEQ ID NO: 58 - RNA, 18 nt, synthetic construct
  source 1..18: mol_type = other RNA
tgcttctctgctcctctc

SEQ ID NO: 59 - RNA, 18 nt, synthetic construct
  source 1..18: mol_type = other RNA
gctgcttctctgctcctc

SEQ ID NO: 60 - RNA, 18 nt, synthetic construct
  source 1..18: mol_type = other RNA
ctgctgcttctctgctcc

SEQ ID NO: 61 - RNA, 18 nt, synthetic construct
  source 1..18: mol_type = other RNA
ttctgctgcttctctgct

SEQ ID NO: 62 - RNA, 18 nt, synthetic construct
  source 1..18: mol_type = other RNA
gcttctgctgcttctctg

SEQ ID NO: 63 - RNA, 18 nt, synthetic construct
  source 1..18: mol_type = other RNA
ctgcttctgctgcttctc

SEQ ID NO: 64 - RNA, 18 nt, synthetic construct
  source 1..18: mol_type = other RNA
ggctgcttctgctgcttc

SEQ ID NO: 65 - RNA, 18 nt, synthetic construct
  source 1..18: mol_type = other RNA
agagcccagaatcctatt

SEQ ID NO: 66 - RNA, 18 nt, synthetic construct
  source 1..18: mol_type = other RNA
cagagcccagaatcctat

SEQ ID NO: 67 - RNA, 18 nt, synthetic construct
  source 1..18: mol_type = other RNA
gcagagcccagaatccta

SEQ ID NO: 68 - RNA, 18 nt, synthetic construct
  source 1..18: mol_type = other RNA
agcagagcccagaatcct

SEQ ID NO: 69 - RNA, 18 nt, synthetic construct
  source 1..18: mol_type = other RNA
gagcagagcccagaatcc

SEQ ID NO: 70 - RNA, 18 nt, synthetic construct
  source 1..18: mol_type = other RNA
tgagcagagcccagaatc

SEQ ID NO: 71 - RNA, 18 nt, synthetic construct
  source 1..18: mol_type = other RNA
ctgagcagagcccagaat

SEQ ID NO: 72 - RNA, 18 nt, synthetic construct
  source 1..18: mol_type = other RNA
tctgagcagagcccagaa

SEQ ID NO: 73 - RNA, 18 nt, synthetic construct
  source 1..18: mol_type = other RNA
ctctgagcagagcccaga

SEQ ID NO: 74 - RNA, 18 nt, synthetic construct
  source 1..18: mol_type = other RNA
actctgagcagagcccag

SEQ ID NO: 75 - RNA, 18 nt, synthetic construct
  source 1..18: mol_type = other RNA
gactctgagcagagccca

SEQ ID NO: 76 - RNA, 18 nt, synthetic construct
  source 1..18: mol_type = other RNA
tgactctgagcagagccc

SEQ ID NO: 77 - RNA, 18 nt, synthetic construct
  source 1..18: mol_type = other RNA
ctgactctgagcagagcc

SEQ ID NO: 78 - RNA, 18 nt, synthetic construct
  source 1..18: mol_type = other RNA
cctgactctgagcagagc

SEQ ID NO: 79 - RNA, 18 nt, synthetic construct
  source 1..18: mol_type = other RNA
gcctgactctgagcagag

SEQ ID NO: 80 - RNA, 18 nt, synthetic construct
  source 1..18: mol_type = other RNA
agcctgactctgagcaga

SEQ ID NO: 81 - RNA, 18 nt, synthetic construct
  source 1..18: mol_type = other RNA
cagcctgactctgagcag

SEQ ID NO: 82 - RNA, 18 nt, synthetic construct
  source 1..18: mol_type = other RNA
gcagcctgactctgagca

SEQ ID NO: 83 - RNA, 18 nt, synthetic construct
  source 1..18: mol_type = other RNA
agcagcctgactctgagc

SEQ ID NO: 84 - RNA, 18 nt, synthetic construct
  source 1..18: mol_type = other RNA
cagcagcctgactctgag

SEQ ID NO: 85 - RNA, 18 nt, synthetic construct
  source 1..18: mol_type = other RNA
tggctgcttctgctgctt

SEQ ID NO: 86 - RNA, 18 nt, synthetic construct
  source 1..18: mol_type = other RNA
ttggctgcttctgctgct

SEQ ID NO: 87 - RNA, 18 nt, synthetic construct
  source 1..18: mol_type = other RNA
cttggctgcttctgctgc

SEQ ID NO: 88 - RNA, 18 nt, synthetic construct
  source 1..18: mol_type = other RNA
tcttggctgcttctgctg

SEQ ID NO: 89 - RNA, 18 nt, synthetic construct
  source 1..18: mol_type = other RNA
ctcttggctgcttctgct

SEQ ID NO: 90 - RNA, 18 nt, synthetic construct
  source 1..18: mol_type = other RNA
gctcttggctgcttctgc

SEQ ID NO: 91 - RNA, 18 nt, synthetic construct
  source 1..18: mol_type = other RNA
agctcttggctgcttctg

SEQ ID NO: 92 - RNA, 18 nt, synthetic construct
  source 1..18: mol_type = other RNA
cagctcttggctgcttct

SEQ ID NO: 93 - RNA, 18 nt, synthetic construct
  source 1..18: mol_type = other RNA
ccagctcttggctgcttc

SEQ ID NO: 94 - RNA, 18 nt, synthetic construct
  source 1..18: mol_type = other RNA
tccagctcttggctgctt

SEQ ID NO: 95 - RNA, 18 nt, synthetic construct
  source 1..18: mol_type = other RNA
ctccagctcttggctgct

SEQ ID NO: 96 - RNA, 18 nt, synthetic construct
  source 1..18: mol_type = other RNA
gctccagctcttggctgc

SEQ ID NO: 97 - RNA, 18 nt, synthetic construct
  source 1..18: mol_type = other RNA
ggctccagctcttggctg

SEQ ID NO: 98 - RNA, 18 nt, synthetic construct
  source 1..18: mol_type = other RNA
tggctccagctcttggct

SEQ ID NO: 99 - RNA, 18 nt, synthetic construct
  source 1..18: mol_type = other RNA
ctggctccagctcttggc

SEQ ID NO: 100 - RNA, 18 nt, synthetic construct
  source 1..18: mol_type = other RNA
tctggctccagctcttgg

SEQ ID NO: 101 - RNA, 18 nt, synthetic construct
  source 1..18: mol_type = other RNA
gtctggctccagctcttg

SEQ ID NO: 102 - RNA, 18 nt, synthetic construct
  source 1..18: mol_type = other RNA
ggtctggctccagctctt

SEQ ID NO: 103 - RNA, 18 nt, synthetic construct
  source 1..18: mol_type = other RNA
tggtctggctccagctct

SEQ ID NO: 104 - RNA, 18 nt, synthetic construct
  source 1..18: mol_type = other RNA
ctggtctggctccagctc